(19) United States
(12) Patent Application Publication
(10) Pub. No.: US 2015/0094609 A1
(43) Pub. Date: Apr. 2, 2015

(54) METHODS AND SYSTEMS FOR A SELF-RETAINING NASAL DILATOR

(71) Applicant: Alan L. Jacobson, Jupiter, FL (US)

(72) Inventor: Alan L. Jacobson, Jupiter, FL (US)

(73) Assignee: Alan L. Jacobson, Jupiter, FL (US)

(21) Appl. No.: 14/499,638

(22) Filed: Sep. 29, 2014

Related U.S. Application Data
(60) Provisional application No. 61/885,448, filed on Oct. 1, 2013.

(51) Int. Cl. A61F 5/08; A61F 13/36; A61F 5/56; A61M 15/08; A61J 15/00; A61B 5/00; A61B 5/01; A61B 5/22; A61M 16/06; A61M 29/02; A61M 16/16

(52) U.S. Cl. CPC A61F 5/08; A61M 29/02; A61F 13/36; A61F 5/56; A61M 15/08; A61M 16/16; A61B 5/6819; A61B 5/01; A61B 5/222; A61M 16/0672; A61J 15/0003; A61J 15/0042; A61F 2210/0061; A61F 2230/0054; A61F 2250/0003; A61M 2205/0216; A61M 2210/0618; A61M 2230/50
USPC 600/549; 606/199; 606/196; 128/848; 128/203.22; 600/300; 604/96.01

(57) ABSTRACT

Methods and systems are provided for a device having a first member and a second member coupled to the first member where the second member is configured to modify a configuration of a human or animal orifice or conduit. In some embodiments, the first member is a shaft and the second member is an inflatable membrane system configured to facilitate inflation or deflation of the inflatable membrane system. In some embodiments, the first member is a U-shaped member with end members configured for insertion to an internal nasal valve. Other embodiments are disclosed.

### CROSS REFERENCE TO RELATED APPLICATIONS

This application claims the benefit of U.S. provisional patent application No. 61/885,448 filed on Oct. 1, 2013, the disclosure of which is incorporated herein by reference in its entirety.

### FIELD

The embodiments herein relate to medical devices and treatments and in particular to systems, devices and modalities utilizing concentric and non concentric balloons, or utilizing solid or semi-solid devices. The devices are intended for human and animal use and are placed within an orifice and/or a physiological/anatomical conduit. The device will support or reshape the anatomical target location or act as a platform to facilitate a medical treatment or to secure a device used for a diagnostic or therapeutic application.

### BACKGROUND

During the action of breathing air passes through the upper airway defined as all structures found in the nose, mouth, pharynx, hypopharynx and larynx. After transitioning through the larynx the airflow enters into the lower airway defined as subglottis trachea, bronchi and other lung components. Many factors affect breathing, one of the most pertinent of which is airway resistance. Common medical knowledge is that conditions causing upper airway resistance are the most common patient complaints as related to breathing conditions.

The nose is responsible for two thirds of the resistance found in the upper airway. The etiologies of nasal resistance can be anatomical, such as seen with septal deviations and cartilaginous deformities or secondary to mucosal abnormalities such as Rhinitis. Regardless of the cause of the obstruction, the gate-keeper of the nasal airway and the lynch pin of nasal resistance is the structure called the nasal valve.

First described by J P Mink in 1903, the nasal valve is divided into an internal and external component. (See FIG. 4 of Internal Valve or “IV”). See Mink J P, Le nez comeme void respiratorie Presse Otolaryngol (Belg) 1963; 21: pages 481-496

In one embodiment of the device, the internal nasal valve is addressed. The internal nasal valve area is formed by the nasal septum (S), the caudalborder of the upper lateral cartilage (UC), the head of the inferior turbinate and the pyriform aperture (PA). (See FIG. 2 & FIG. 3).

The internal nasal valve by definition is usually the narrowest part of the nasal airway and contributes to 70% of nasal resistance. An excellent description of the nasal valve and its functions are described by Cole. See Cole, “The Four Components of the Nasal Valve” American Journal of Rhinology Vol. 17 No 2 pp. 107-110 (2003).

Persons who have difficulty with nasal breathing can experience profound changes in quality of life, which can be objectively quantified by the Nasal Obstruction Symptom Evaluation scale (NOSE). These problems can include sleep disturbance and apnea, snoring, nasal dryness, recurrent Epistaxis, decreased physical performance, an d decreased sexual performance and multiple of other symptoms.

Modalities to correct nasal valve abnormalities and thus improve airway, can be divided into two groups, surgery and nasal devices.

Surgery is costly, carries risks and is not always successful. Nasal devices can be classified as external and internal nasal expanders. The concept behind expanders is to enlarge the internal nasal valve by placing a force (internal or external) upon one or more components, which automatically define or affect the valve.

The caudal most part of the internal valve is an angle between the septum and the caudal border of the upper lateral cartilage. This angle is usually about 10-15°. The cross-sectional area of the internal nasal valve is normally 55-83 mm². As described by Poiseuille's Law, small changes in the radius of the narrowest portion of the nose will cause a dramatic and exceptional increase in airway resistance in the nose and entire respiratory system. Therefore enlarging the area of the internal valve will decrease resistance and the work of breathing.

External nasal expanders are currently the most widely used devices on the market today. The Breath Right Strip™ is the best example of this kind of device, see U.S. Pat. No. 5,533,499 to Johnson, U.S. Pat. No. 5,533,503 to Doubek et. al, and U.S. Pat. No. 7,114,495 to Lockwood, Jr. External expanders are not uniformly successful and their efficiency has been shown to be diminished in non-Caucasian individuals. Additionally they may be dislodged during sleep and the adhesive can be irritating to the skin especially with continued usage. (FIGS. 1A, 1B, and 1C)

Internal nasal expanders are placed inside of the nose and include clips, spring and cylindrical stints such as Max Air Nose Cones© U.S. Pat. No. 7,055,523, U.S. Pat. No. 6,978,781. Problems with internal expanders are much more prolific and are primarily centered around a foreign body sensation and discomfort causing decreased compliance. Additionally the devices are not adjustable to fit the variety of nose shapes and anatomical variations.

### BRIEF DESCRIPTION OF THE FIGURES

The embodiments and the following detailed description of certain embodiments thereof may be understood by reference to the following figures:

FIGS. 1A, 1B, and 1C illustrate the variations of nasal anatomy by race or ethnicity which can have significant differences.

FIG. 1A illustrates an African nasal anatomy which is the widest width and is shorter then the Caucasian nose. The African nose has the least triangular shaped nasal base and widest piriform aperture, flattest tip and largest internal nasal valve area.

FIG. 1B illustrates an Asian nasal anatomy which is intermediate in shape and proportion compared to the Caucasian and African nose.

FIG. 1C illustrates a Caucasian nasal anatomy with a larger nasal spine, thinner skin and more cartilage support than the other variations. The alar width is narrow and piriform aperture is narrow and nostril is vertical in position

FIG. 2 illustrates a partial perspective view of the external bony and cartilaginous anatomy. Looking at the left side of the nose we see the nasal bone (NB), the septum (S) which separates the nose into right and left sides and makes up the medial wall of the internal nasal valve. The upper lateral (UC) and lower lateral (LC) cartilages as well as the nostril (N) and alar (A) areas (see FIG. 3) of the nose are shown.

FIG. 3 illustrates the base view of the cartilaginous anatomy and their relationship to the internal nasal valve.

FIG. 4 illustrates the position of the internal nasal valve as seen from a nasal base view. The internal valve is an area within the nose the apex of which forms an angle of 10°-15° in most Caucasian noses.

FIG. 5A illustrates a partial perspective view of the external bony and cartilaginous anatomy.

FIG. 5B illustrates the position of a device in the nasal valve in accordance with the embodiments relative to the nasal cartilaginous anatomy.

FIG. 6A is a perspective view of a bilateral balloon in accordance with the embodiments.

FIG. 6B illustrates a top plan view of the bilateral balloon of FIG. 6A.

FIG. 6C is a side view of the bilateral balloon of FIG. 6A.

FIG. 6D is another top plan view of the bilateral balloon of FIG. 6A.

FIG. 7 is an illustration of bilateral balloons in accordance with the embodiments.

FIG. 8A is an illustration of a cotton swab tip inserted into a “U”-shaped device in accordance with the embodiments.

FIG. 8B is an illustration of a cotton swab tip in a U shape device in an integrated form in accordance with the embodiments.

FIG. 8C is an illustration of a cotton swab in a U shaped device having a tube and valves in accordance with an embodiment.

FIG. 8D is an illustration of a U shaped device having a balloon within a balloon in accordance with an embodiment.

FIG. 9A illustrates a single alar clip and balloon arrangement in accordance with an embodiment.

FIG. 9B illustrates a single alar clip and balloon arrangement using a tube and valve in accordance with an embodiment.

FIG. 9C illustrates the use of the single alar clip and balloon in the left nostril and more specifically within the nasal internal valve of a human in accordance with an embodiment.

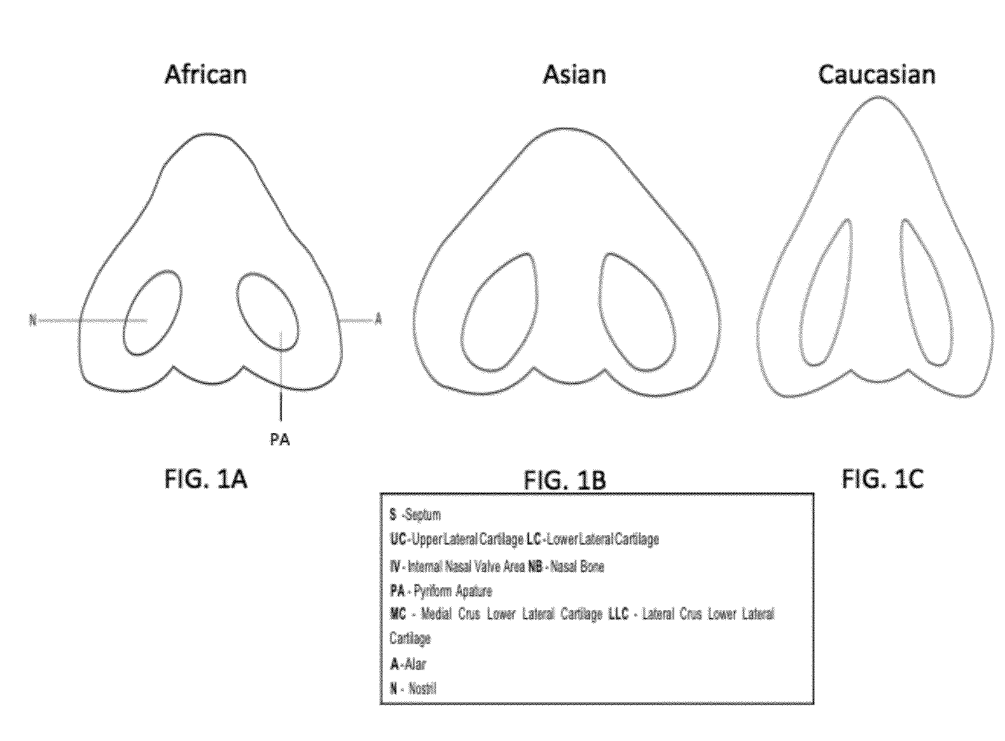

All documents referenced herein are hereby incorporated by reference.

### SUMMARY

In some embodiments, methods and systems are provided for a device intended for human or animal use that can be placed within an orifice and/or a physiological/anatomical conduit and which is based upon an optimally shaped solid, semi-solid compressible material or balloon apparatus or a combination thereof that can pass through or remain within a particular desired anatomical location. In some embodiments, the device includes a first member and a second member coupled to the first member where the second member is configured to modify a configuration of a human or animal orifice. Such a device can facilitate configuring the device to optimally match the anatomy of a target anatomical location discriminated by variables such as gender, ethnicity, age, and trauma and further facilitate reshaping of desired and specific parts of human and or animal anatomy. In some embodiments, the device facilitates occlusion or dilatation of orifices or conduits in humans or animals, and for removal of obstructing objects and for better visualization of anatomical structures for the purpose of evaluation and treatment. Some of the human or animal orifices or conduits can include the nasal passage or passages, sinuses, cardiovascular structures, or gastrointestinal structures.

The device can be based on uniquely shaped balloons or other implementations that can be concentric or eccentric, which are placed within human or animal subjects. Each shape can be optimized for a particular target anatomical structure or location. Note that the shapes are not necessarily limited to concentric or eccentric shapes and not limited to fluid or air-filled balloons. For example, the shapes can be variable and can further include spherical, non-spherical, bean-shaped, kidney bean shaped, compressible, non-compressible, porous, non-porous, synthetic, and natural fibers. Other shapes are contemplated within the scope of the claimed embodiments herein. The implementations can be solid and can include synthetic plastics or rubber. Some implementations can be semi-solid and can include for example cotton swabs or absorbent fibers with varying tightness in weave. In some implementations, a balloon can be used that can be made of rubber, butyl or other plastics known in the art. In some implementations, the device is used to decrease air resistance and turbulence through the nasal passage. In some embodiments, the device can include a shaft member and a second member having an inflatable membrane system coupled to the shaft member. The shaft member and second member can be configured to facilitate inflation or deflation of the inflatable membrane system at a desired anatomical location.

The device can remain in place for short as well as extended durations. If the device is intended to remain in place for extended durations, the device can be coated with antiseptic particles or an antiseptic layer to avoid infections.

The device can support or reshape anatomy to facilitate a specific desired goal. Such goals can include and are not limited to improved breathing, improved air flow, removal of obstructing objects, better visualization of anatomical structures for the purpose of evaluation and treatment, improved delivery of medications through anatomical cavities such as nasal passages.

The device can act as a platform to facilitate securing other devices. For example, the device can be used in surgical procedures or exploratory procedures and can temporarily fix a camera, a drill, a laser, a suction tube or other tool using compression against a conduit wall.

The device can be reusable and can be combined with other devices to facilitate different uses. In some embodiments, the device can be disposable or single use device. In some embodiments, the device can include a substantially U-shaped shaft member having a first opposing distal end and a second opposing distal end and end members coupled to the respective first and second opposing distal ends. The end members can be configured for insertion to an internal nasal valve.

### DETAILED DESCRIPTION

The features of the present embodiments, which are believed to be novel, are set forth with particularity in the appended claims. The embodiments can best be understood by reference to the following description, taken in conjunction with the accompanying drawings.

While the specification concludes with the claims defining the features of the embodiments that are regarded as novel, it is believed that the embodiments may be better understood from a consideration of the following description in conjunction with the drawings figures, in which like reference numerals are carried forward.

The terms and phrases used herein are not intended to be limiting but rather to provide an understandable description of the embodiments.

The terms “a” or “an”, as used herein, are defied as one or more than one.

The term “another”, as used herein, is defined as at least a second or more. The terms “including” and/or “having” as used herein, are defined as comprising (i.e. open transition). The term “coupled” or “operatively coupled” as used herein, is defined as connected, although not necessarily directly, and not necessarily mechanically.

The embodiments herein can be reusable disposable devices that could be used independently or in conjunction with external expanders. Unlike other internal devices, some of the embodiments herein are adjustable to conform to different nasal anatomies and is effective for different racial anatomical variations. The embodiments further address many conditions related to the nose and upper airway as well as a new and unique modality to secure devices which pass through the nose to treat and evaluate a variety of medical conditions.

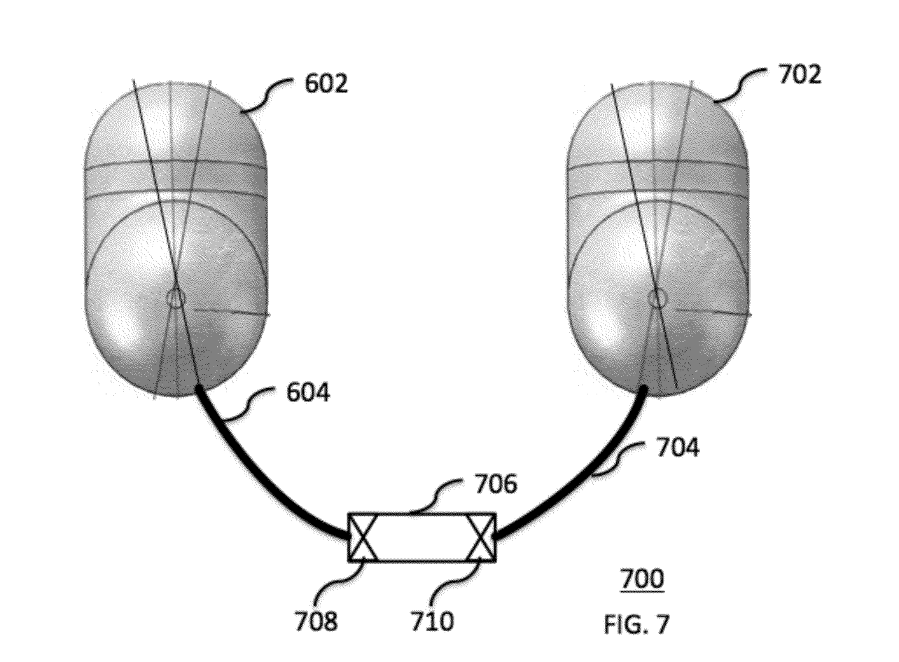

In some embodiments, the balloon device has a unique non-concentric design that is adaptable for endoscopic and endovascular, endo parenchymal, endo vesicular, inter and intra osseous, diagnostic and therapeutic medical procedures. (see FIGS. 7-9)

In some embodiments, methods and systems are provided for a medical device where its embodiments are intended to be a platform for a self-retaining nasal instrument, which is adaptable to provide multiple functions within the nose based upon unique non-concentric and concentric balloon and non-balloon designs.

In some embodiments, a device may be placed into one nostril and in other embodiments the device can be placed bilaterally or in both nostrils. (see FIG. 7). In one example, the bilateral device can be a substantially U-shaped device that can be made of a single unitary shaft construction. The U-shape can be a smooth single curved shape or formed from a horizontal support strut and two opposing substantially vertical struts or members. The horizontal support strut and the respect vertical struts can be formed at obtuse angles to provide an outward lateral force when placed within a pair of nostrils and within respective internal nasal valves. The end members of the U-shaped device can simply include a cotton swab or other absorbent material. In some embodiments the end members can include balloons as shown in FIG. 7 and in yet other embodiments the end members can include a solid or semi-solid member that can be conveniently inserted and removed from the internal nasal valve area. In some embodiments, the semi-solid member can be similar to a cotton swab tip. In some embodiments, the end members such as a balloon is positioned or rotated approximately 10% off the midline of the balloon or end member to facilitate the lateral and superior distortion of the nasal valve when the end member is placed within the nostril (and nasal valve). In other words, the end member causes the nasal valve to move outward and upwards when the end member is inserted in the nostril and within the nasal valve area.

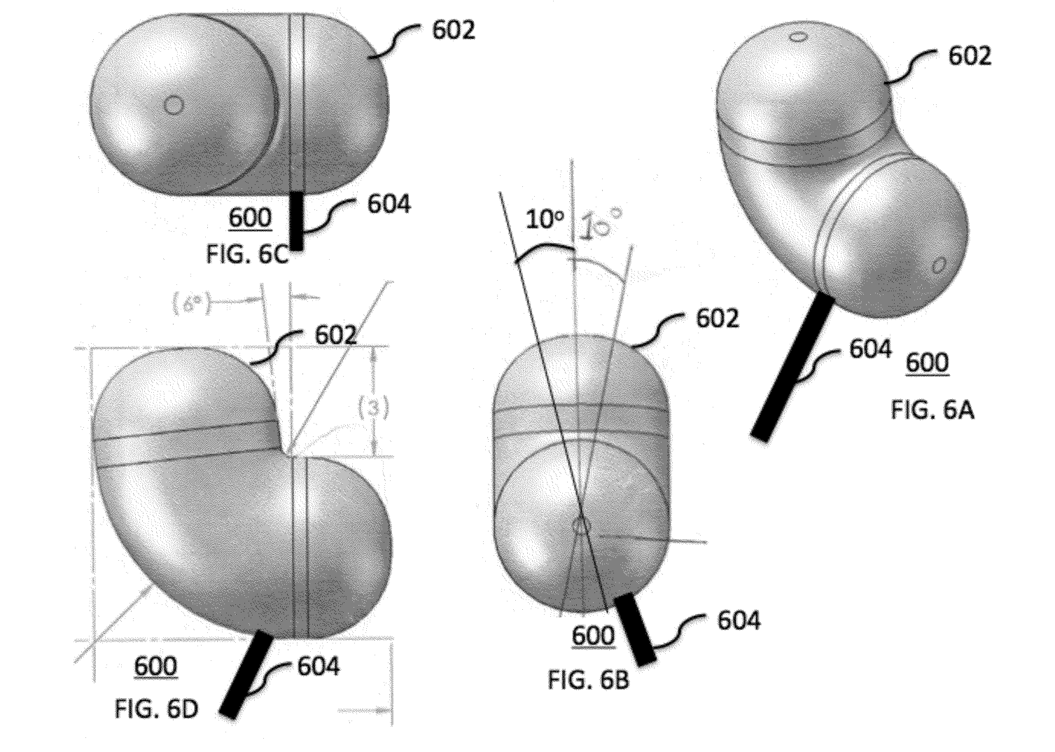

FIG. 6A illustrates a perspective partial view of a bilateral balloon system 600 including a left balloon 602 and a tubular structure 604. Note that only a portion of the tubular structure 604 is shown in FIGS. 6A-D. FIG. 6B illustrates a top view of the left balloon 602 where the tubular structure is positioned approximately 10% off of the midline of the balloon 602 to facilitate the lateral distortion of the nasal valve as discussed above. FIG. 6C is another top view of the left balloon 602 and the tubular structure 604. FIG. 6D is a side view of the left balloon 602 and the tubular structure 604.

In one embodiment as shown in FIG. 7, a bilateral balloon system 700 includes a first balloon 602 that is eccentrically shaped (such as a kidney shape) that is fitted over an open cell foam piece that sits at the distal end of a short hollow tubular structure 604 as shown as well as a second balloon 702 (similarly shaped as balloon 602) that is fitted over another short hollow tubular structure 704. In some embodiments, the tubular structure 604 and 704 can be one and the same tube. In other embodiments, the tube 604 and 704 can include a small valve. In yet other embodiments, the respective tubes 604 and 704 can couple to a valve module 706 having respective valves 708 and 708 that allow air or fluid into the respective balloons to inflate the balloons. For example, the valve module 706 (or tube) can contain at least a small valve (or two valves) such as a duckbill valve which when compressed will allow air or fluid into or out of the balloon. The valve or valves can also alternatively allow the valve module 706 to operate as a pump for inflation or deflation of the respective balloons. The tube also allows the device to be placed within the nose specifically within the area of the internal nasal valve. The device is placed while the foam is compressed and the balloon is deflated. Once in position, the valve is compressed and the air will displace the vacuum in the balloon to allow the foam to expand. Multiple size balloons of different shapes are available and therefore the device is self-adjustable and can fit all varieties of noses—both human and animal.

Some embodiments relate to medical devices and treatments, and in particular to systems, devices and modalities for treating the nose and conditions related to the nose and upper airway. The embodiments further address modalities to secure devices which pass through the nose to treat a variety of medical conditions. Some of the devices that pass through the nose can include a nasal endoscope or speculum to name a few.

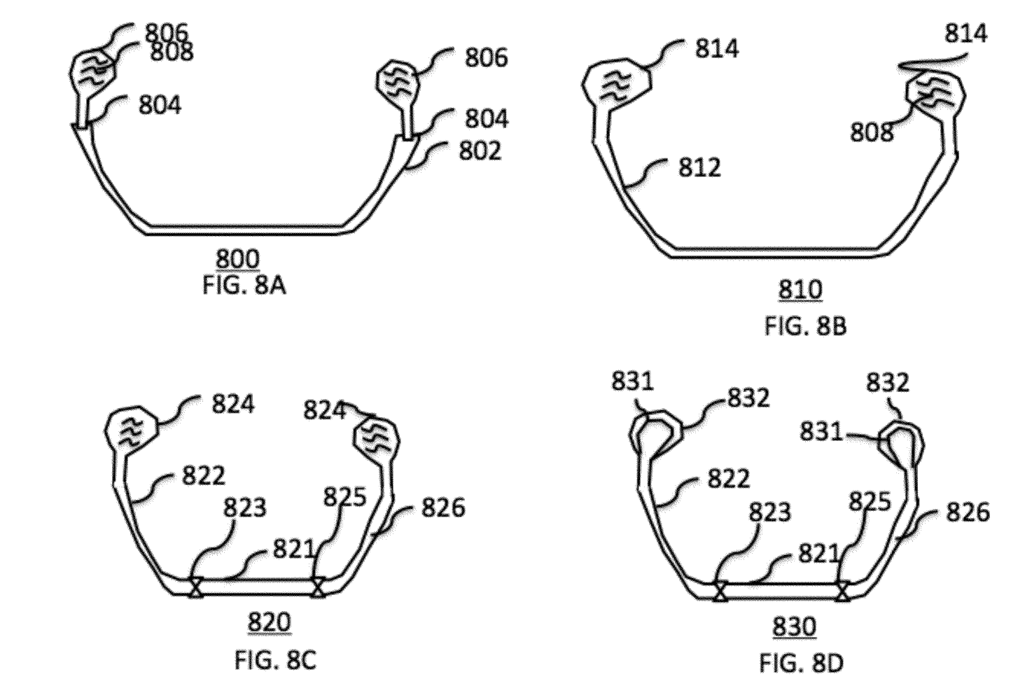

An embodiment of the device may reduce resistance to nasal and upper airway airflow and therefore improve airflow or subjective feeling of airflow during breathing. Thus, a simple u-shaped device as shown in FIG. 8A, 8B, or 8C can be used to alleviate and improve breathing during sleep or during physical exercise. FIG. 8A illustrates a U-shaped device 800 having a first member 802 and a second member 806 coupled to the first member 804 where the second member 806 is configured to modify a configuration of a human or animal orifice such as a nasal valve. In some embodiments, the first member is a stick and the second member is an expandable membrane or balloon. In some embodiments, the balloon can be a non-concentric balloon (as previously shown in FIGS. 6 and 7). Design of the non-concentric balloon can configured to self retain within a human or animal orifice such as the internal nasal valve area. The balloon can also be designed to facilitate insertion and removal to the internal nasal valve. In some other embodiments, the first member is a substantially u-shaped member and the second member is at least one cotton swab at a distal end of the substantially u-shaped member. In FIG. 8A, the first member 802 has a substantially u-shaped member having two opposing distal ends 804 and the second member comprises a cotton swab 806 respectively at each opposing distal end of the U-shaped member.

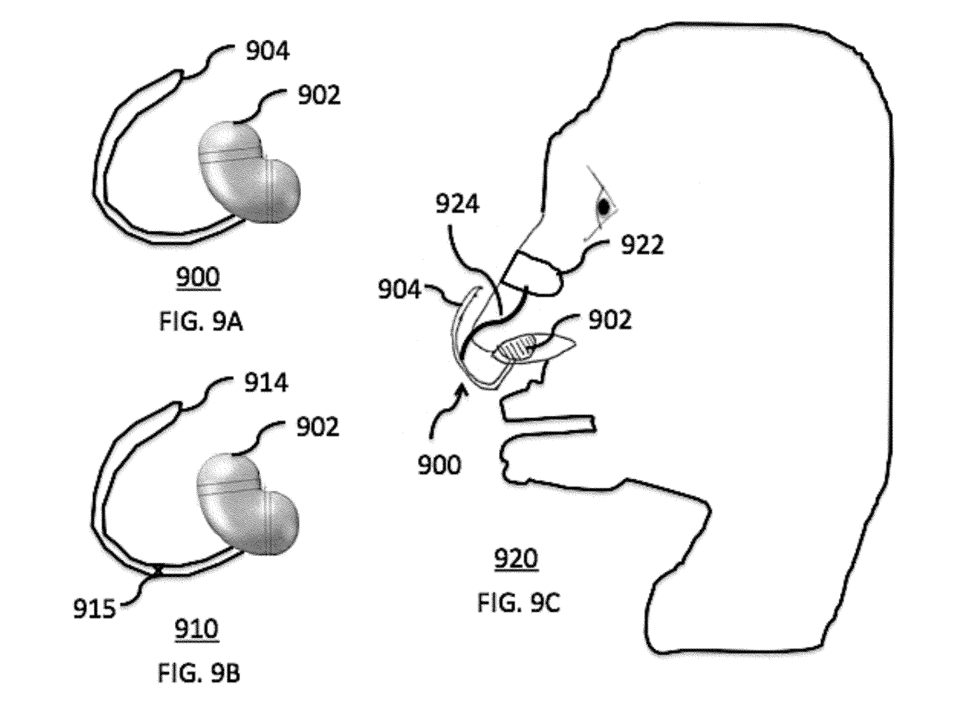

In some embodiments, the tubular member 802 can be configured to convey a fluid or a gas to the second member for selective inflation (or deflation) of the second member. The fluid or gas can contain some form of medicament or the medicine 808 can be embedded in a cotton swab as shown in FIGS. 8A and 8B or the medicine can be contained in the tubes or reservoirs (see 821 or 822 or 826 of FIG. 8C or 8D). Thus, the reservoir or reservoirs in the various embodiments herein can be used as a repository for a medicated substance which is then delivered to the distal end (e.g., 806, 814, 824, or 832) of the device via an external force or via capillary action for example. In a slight variation, medicament in the form of coatings on the surface of the inflatable membrane system or through trans mural permeability of a balloon wall of the inflatable membrane system can be used to deliver such medicines. Further note that the device can be used and/or coupled to an external nasal expander (see FIG. 9C) where the external nasal expander is coupled to device (700, 800, 810, 820, 900, 910 or 920 as shown) and to the first member (904) of the device 920.

The U-shaped device 800 as shown in FIG. 8A can be configured to have a U-shaped member 802 with respective holes or receptacles for receiving and inserting the second member 806 which can be a cotton swab or balloon. In some embodiments as illustrated in FIG. 8B, a U-shaped device 810 can include a U-shaped member 812 integrated with the end member 814 (such as a cotton swab). The cotton swab 814 can optionally include medicine or some form of medicament 808 embedded in the cotton swab 814. In yet other embodiments, a U-shaped device 820 as shown in FIG. 8C can include an integrated U-shaped member and cotton swab 824 where the U-shaped member is formed of one or more tubes such as tubes 821, 822 and 826. In some embodiments, the tubes can include small valves 823 and 825 as shown to enable flow of gas or fluid (or same containing medicine) to or from the end members 824. In yet some other variants but similar to the embodiments of FIG. 8A, 8B or 8C, the U-Shaped member can couple or be integrated with one or more balloons. For example, in the embodiment shown in FIG. 8D, a device similar to device of 820 of FIG. 8C includes an internal balloon 831 within an external balloon 832 as an end member instead of a cotton swab.

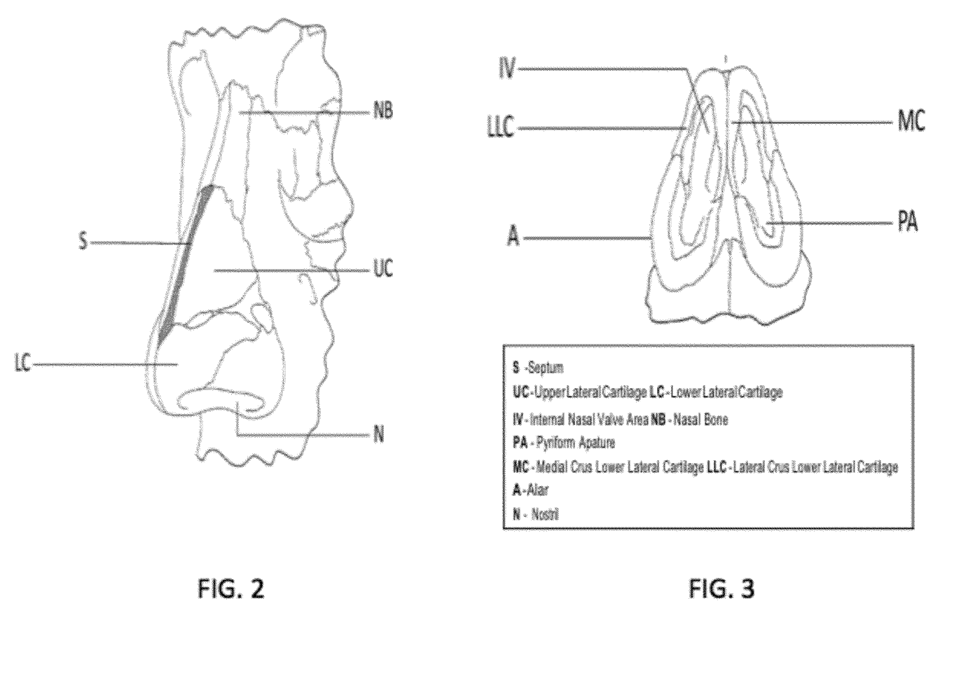

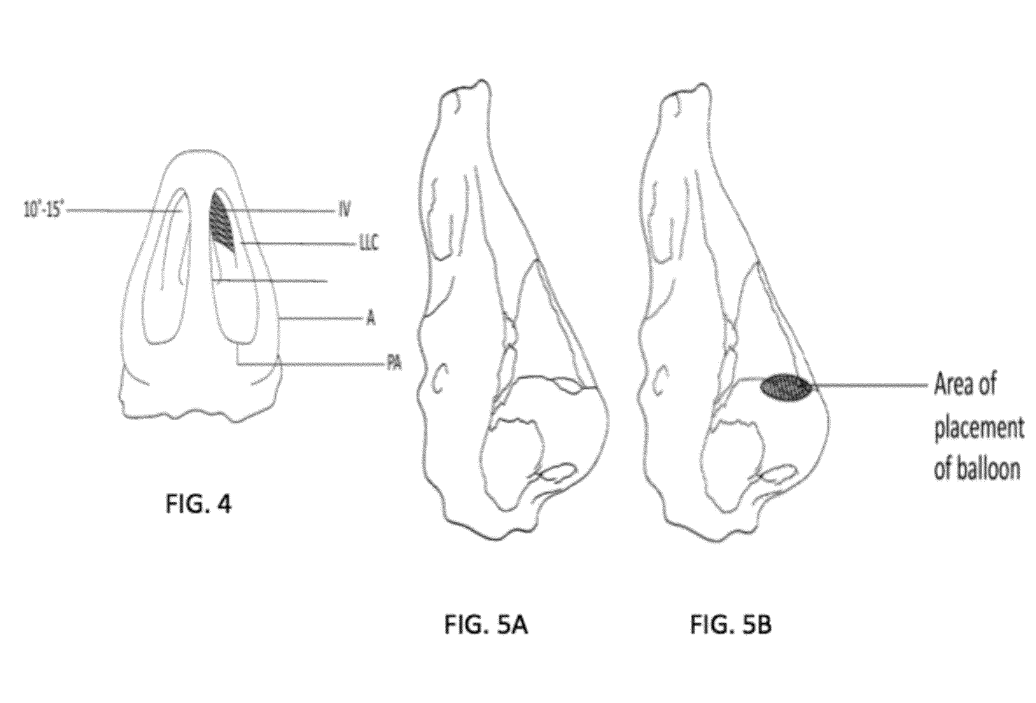

The device displaces the upper lateral cartilage (ULC) laterally and the skin between the cutaneous and respiratory mucosa superiorly. The displacement increases the angle of the internal nasal valve (ne 10-15°) as well as the nasoseptal angle. The action of this displacement will decrease the resistance to breathing and provide better airflow through the nasal passages. (FIGS. 2-5)

In another embodiment the balloon is intended for both nostrils and a hollow tube which connects to each balloon is connected to a horizontal tube which contains the inflating or deflating valve. The angle between the horizontal and vertical tubes can be obtuse so that when the balloons are inflated there is additional lateral force applied to the internal nasal valve (this differs from existing devices where the force is applied to the septum).

In one embodiment of the device there may be a spring material that is part of the hollow tube struts that serves to increase the lateral force applied to the valve area. The spring material can be the plastic or paper or pulp product itself that is biased with an outward tension such that a lateral force is applied when the device is inserted in the nasal valve area.

To remove the balloons, the balloons are displaced downward and taken out of the nose and deflated. The balloons can be compressed to a certain extent before the downward displacement to help avoid discomfort in removal of the device.

### EXAMPLE

This example is intended to change the angle of the internal nasal valve and thus decrease resistance in breathing.

As the device in certain embodiments is a self retaining adjustable instrument, it may serve as the platform for a variety of other devices to provide the following exemplary treatments and functions:
a. Localized Epistaxis (nose bleed) Control
b. Nasal Humidification
c. Nasal Thermometer
d. Pediatric Self Retaining 02 Delivery System
e. Pediatric Self Retaining Ng Tube Holder
f. Sleep Apnea Evanston on Monitoring Screening Device
g. Sleep Apnea Treatment
h. Nasal Pulse Oximeter—Wireless or Hardwired
i. Treatment of Snoring
j. Treatment of Rhinitis of Pregnancy
k. Intra Nasal Delivery System for medications
l. Device to improve and monitor athletic performance
m. Adult self retaining device for nasal 02 and Ng tubes
n. Collapse of Nasal Valve
o. Nasal Obstruction

In an embodiment, a balloon is filled with semi-viscous liquid which is stored in one or two reservoirs located within the horizontal strut of the device or in any other reservoir designed for use with the device. To inflate or deflate the balloons the valves are compressed and the reservoirs are emptied via compression or refilled by compressing the balloon. Examples of fluids that can be used include, but are not limited to, water, saline, gel, silicon, or oil. The fluid should generally be non-toxic for human and animal uses.

In some embodiments, two balloons may be placed in each nostril, where a first balloon may be placed within a second balloon as previously discussed and shown with respect to FIG. 8D. The internal balloon can be in a permanent state of inflation and the other balloon is connected to the reservoir. Thus, the external balloon provides variability for a number of different shaped nasal or other anatomical structures among different patients.

In some embodiments of this design there is a U shaped clip attached to either the vertical or horizontal member of this device. This clip may attach to either the nasal columella or the nasal alar. The clip will apply a gentle compressive force which could act to further secure the balloon device. For example, the embodiment of FIG. 9A illustrates a device 900 having a alar clip 904 coupled to a balloon 902. Similarly, in FIG. 9B, a device 910 includes an alar clip 914 and a balloon 902 except that the clip can be in the form of a tube and can include a valve 915 to compress fluid or air towards or from the balloon 902. Further note that in some embodiments, the balloon 902 can include an internal balloon or internal body and an external balloon. The device 900 can be used alone as a single clip for a single nostril or can be used as a pair for separate insertion in separate nostrils. As illustrated in FIG. 9C, a system 920 can include the device 900 operating in conjunction with an external nasal expander 922 and in some embodiments the device 900 can be coupled to the external nasal expander 922 via a string or elastic member 924.

Some embodiments of this device utilize the unique self-retaining properties to act as method to secure tubes, wires or other devices which must pass through the nose. The balloon application fixates these other devices or items so they may not fall out of the nose or become inadvertently dislodged from the nose. The balloon application can be used for fixing a nasal feeding tube for example.

In other embodiments of this device the balloon system allows for the selective and precise delivery of nasal medications to pre selected areas of the nasal cavity. The nasal medications may be used independently or in combination with a compressive force caused by the balloon. This application may be used for a variety of treatment applications for the nose including but not limited to control of Epistaxis (nose bleeds) and treatment of nasal congestion. Note that in some embodiments, the balloon system (or a non-balloon system) can be combined with existing trans mucosal or transcutaneous medication delivery techniques. In some embodiments, the balloon or solid tip, or semi-solid tip can be embedded with medications or vitamins. For example, the tip can include a coagulant for controlling Epistaxis or with vitamin B-12 for treating vitamin B-12 deficiency.

Additional embodiments of this device consist of a nasal humidification system where by a humificant can be selectively applied to the nose in a non-aerosolized manner.

Other embodiments allow the balloon system to act as a platform for transmucosal and transcutaneous nasal oximetry devices. For example, embodiments herein can be used for an efficient measurement of PO2 (pulse oximetry). This is particularly relevant in intra operate situations as well as part of a system used to evaluate and monitor sleep apnea, sudden infant death syndrome (SIDS) and athletic performance for therapeutic and training purposes.

Other embodiments of this device are intended to occlude the nostril, when combined with a specific one way valve, the device may act to increase the upper airway pressure in such a manner needed to overcome and improve the airway collapse and resistance associated with sleep apnea.

Another embodiment of this device is a balloon or solid core device or a semi-solid device which is enveloped by an absorbent or porous material which can be used to deliver an aromatic or volatile substance to the nasal passage or passages. The shape and design of the device would self retain within the nose as well as open and expand the internal nasal valve. The device is placed on the distal end of a small rigid or semi-rigid stick or string which aids in placement, retention and removal of the device. In one embodiment, the device can be combined with an external nasal expander (similar to the Breathe Right™ device) where a string can be attached between the external expander and a stem of the device that would be placed towards the internal nasal valve as shown in FIG. 9C.

In another embodiment of this device this unique non-concentric shape of the balloon device allows the placement of the device in an orifice or conduit or other body structure to facilitate diagnostic or therapeutic treatments in areas of the body other than the nose.

It will be appreciated that the various steps identified and described above may be varied, and that the order of steps may be adapted to particular applications of the techniques disclosed herein. All such variations and modifications are intended to fall within the scope of this disclosure. As such, the depiction and/or description of an order for various steps should not be understood to require a particular order of execution for those steps, unless required by a particular application, or explicitly stated or otherwise clear from the context.

While the invention has been disclosed in connection with the preferred embodiments shown and described in detail, various modifications and improvements thereon will become readily apparent to those skilled in the art. Accordingly, the spirit and scope of the present invention is not to be limited by the foregoing examples, but is to be understood in the broadest sense allowable by law.

All documents referenced herein are hereby incorporated by reference.

I claim:

1. A device comprising:
a first member;
a second member coupled to the first member, the second member configured to modify an configuration of a human or animal orifice.

2. The device of claim 1, wherein the first member is a stick and the second member is an expandable membrane or balloon.

3. The device of claim 1, wherein the first member is a substantially u-shaped member and the second member comprises at least one cotton swab at a distal end of the substantially u-shaped member.

4. The device of claim 1, wherein the first member is a substantially u-shaped member having two opposing distal ends and the second member comprises a cotton swab at each opposing distal end of the U-shaped member.

5. The device of claim 1, wherein the first member is a tubular member configured to convey a fluid or a gas to the second member for selective inflation of the second member.

6. The device of claim 1, wherein the first member is a tubular member configured to convey a fluid or a gas to the second member for selective inflation or deflation of the second member.

7. The device of claim 5, wherein the second member is a balloon.

8. The device of claim 5, wherein the second member is a non-concentric balloon configured for self-retaining placement within the human or animal orifice.

9. The device of claim 8, wherein the human or animal orifice is an internal nasal valve area and wherein the device is used for or treatment of one of epistaxis control, collapse of nasal valve, nasal obstruction, nasal humidification, sleep apnea, snoring, rhinitis of pregnancy, intra nasal delivery of medications, monitoring of athletic performance, self retaining of nasal 02 or Ng tubes, or as a nasal thermometer.

10. The device of claim 1, wherein the second member is made of a compressible or semi-compressible material.

11. The device of claim 1, wherein the second member is configured to facilitate removal and insertion to an internal nasal valve.

12. The device of claim 1, wherein the second member is configured to provide a lateral force to the internal nasal valve.

13. The device of claim 1, further comprising an external nasal expander coupled to the first member.

14. A device comprising:
a first shaft member;
a second member having an inflatable membrane system coupled to the first shaft member, the first and second members configured to facilitate inflation or deflation of the inflatable membrane system at a desired anatomical location.

15. The device of claim 14, wherein the inflatable membrane system comprises a combination of a balloon and an open cell foam member that facilitates inflation of the balloon to conform to a structure at the desired anatomical location.

16. The device of claim 14, the first shaft member comprises a tubular stent for placement inside of a single nostril or inside of two nostrils of a nose.

17. The device of claim 14, wherein the first shaft member is an elongated shaft having the second member in the form of a balloon at the distal end of the elongated shaft, and wherein the balloon is configured for placement in an internal nasal valve and when inflated, the balloon self retains and changes an angle of the nasal valve for improved laminar airflow through breathing passages.

18. The device of claim 14, wherein the first shaft member comprises a substantially U-shaped member comprising a horizontal strut having opposing ends and substantially vertical struts formed with obtuse angles from the horizontal strut at each of the opposing ends of the horizontal strut to increase a lateral force applied the desired anatomical location.

19. The device of claim 18, wherein the horizontal strut further comprises a reservoir valve system to inflate the inflatable membrane system.

20. The device of claim 14, wherein the inflatable membrane system is configured to facilitate delivery of a medicament comprising coatings on the surface of the inflatable membrane system or through trans mural permeability of a balloon wall of the inflatable membrane system.

21. A device comprising:
a substantially U-shaped shaft member having a first opposing distal end and a second opposing distal end; and
end members coupled to the respective first opposing distal end and second opposing distal end of the U-shaped shaft member, the end members configured for insertion to an internal nasal valve.

22. The device of claim 21, wherein the end members comprise respective cotton swabs.

23. The device of claim 21, wherein the end members comprise respective balloons or elastic membranes.

24. The device of claim 23, wherein the substantially U-shaped shaft member comprises a tubular portion having reservoir valve and wherein fluid or gas is pressured to inflate the respective balloons or elastic membranes on the end members.

25. The device of claim 21, wherein the end members are configured to provide a force to the internal nasal valve in both a lateral direction and a superior direction when the end members are inserted in the internal nasal valve.

26. The device of claim 21, further comprising a nasal expander coupled to the substantially U-shaped shaft member.
